(19) United States
(12) Patent Application Publication
(10) Pub. No.: US 2009/0165784 A1
(43) Pub. Date: Jul. 2, 2009

(54) LUBRICIOUS INTUBATION DEVICE

(75) Inventor: Daniel Holtz, Chittenango, NY (US)

Correspondence Address: TYCO HEALTHCARE - EDWARD S. JARMOLOWICZ, 15 HAMPSHIRE STREET, MANSFIELD, MA, 02048, US

(73) Assignee: TYCO HEALTHCARE GROUP LP, Mansfield, MA (US)

(21) Appl. No.: 11/966,785

(22) Filed: Dec. 28, 2007

(51) Int. Cl. A61M 16/04

(52) U.S. Cl. 128/200.26

(57) ABSTRACT

An intubation device for intubation of a patient that includes a tube comprising a body comprising a mixture of a polymer composition and a lubricant that provides inherent lubricity to the surface of the body and methods for intubating a patient with such a device. The lubricant assists in withdrawal of a stylet wire and does not require activation.

### TECHNICAL FIELD

This invention relates to intubation devices and, more particularly, to lubricious intubation tubes that facilitate removal of a stylet wire without the need to activate a lubricant at the surface of the tube, for example, by contact with water or an alkaline solution.

### BACKGROUND OF THE INVENTION

It is often medically necessary to place a tube in a hollow organ of a patient such as the trachea or stomach. The tubes may be used to provide oxygen to the lungs or provide nutrition to the stomach. To install the tube, the clinician inserts the distal end of the tube through a bodily orifice, such as the mouth or nasal cavity, and feeds the tube through the appropriate body conduits (e.g., larynx, trachea or esophagus). For instance, during a nasal intubation, the tube is continually fed through the nose, past the pharynx and down the esophagus and into the stomach.

To facilitate insertion of the tube into the body, the tube is often inserted with a removable stylet received within the tube body. The stylet provides rigidity to the tube body during insertion and aids in guiding the tube throughout the body conduits. The stylet is removed from the tube once the tube is fully inserted.

Typically and especially in nasogastric intubation procedures, the inner surface of the tube is coated with a hydromer or other coating to reduce friction between the tube and stylet as the stylet wire is withdrawn from the tube. Once the tube is in proper position in the patient, water or an alkaline solution is introduced into the tube to activate the hydromer or coating. The stylet wire is then withdrawn from the tube and the tube can then be used for its intended purpose. The inner surface of the tube must be coated with the hydromer or coating during manufacture of the tube or just prior to the medical procedure.

Thus, a need exists for intubation devices that include a tube with a lubricious inner surface that does not require activation and do not require application of a coating to the inner surface of the tube. Further, there is a need for methods for intubating a patient that do not require activation of a lubricant.

### SUMMARY OF THE INVENTION

In one aspect, an intubation device for intubation of a patient comprises a tube comprising a body that forms a lumen. The body comprises a mixture of a polymer composition and lubricant. The lubricant provides inherent lubricity to the surface of the body. The device comprises a stylet comprising a stylet wire adapted and sized for insertion into and removal from the lumen. The stylet wire provides rigidity to the tube upon insertion into the lumen.

In another aspect, an intubation device for intubation of a patient comprises a tube comprising a body that forms a lumen. The tube is sized to deliver nutrients, water or air from a source external to a patient, through a body orifice and into a body organ of the patient. The body comprises a mixture of a polymer composition and lubricant. The lubricant provides inherent lubricity to the surface of the body. The polymer composition and lubricant are suitable for use in the human body.

In a further aspect, a method for intubating a patient comprises inserting an intubation device through a body orifice and into a body organ of a patient. The device comprises a tube comprising a body that forms a lumen. The body comprises a mixture of a polymer composition and a lubricant. The lubricant provides inherent lubricity to the surface of the body. The device comprises a stylet comprising a stylet wire received within the lumen having a proximal end and a distal end. The stylet wire is removed from the lumen by applying a pulling force to the proximal end of the stylet wire to slide the stylet wire in a proximal direction relative to the tube.

Other features will be, in part, apparent and, in part, pointed out hereinafter. Various refinements exist of the features noted in relation to the above-mentioned aspects of the present invention. Further features may also be incorporated in the above-mentioned aspects of the present invention as well. These refinements and additional features may exist individually or in any combination. For instance, various features discussed below in relation to any of the illustrated embodiments of the present invention may be incorporated into any of the above-described aspects of the present invention, alone or in any combination.

### BRIEF DESCRIPTION OF THE DRAWINGS

FIG. 1 is a perspective of a stylet and intubation tube with the stylet wire partially inserted into the tube;

FIG. 2 is a cross-section of the stylet shown in FIG. 1; and

FIG. 3 is a cross-section of an alternate stylet.

Corresponding reference characters indicate corresponding parts throughout the drawings.

### DETAILED DESCRIPTION OF THE DRAWINGS

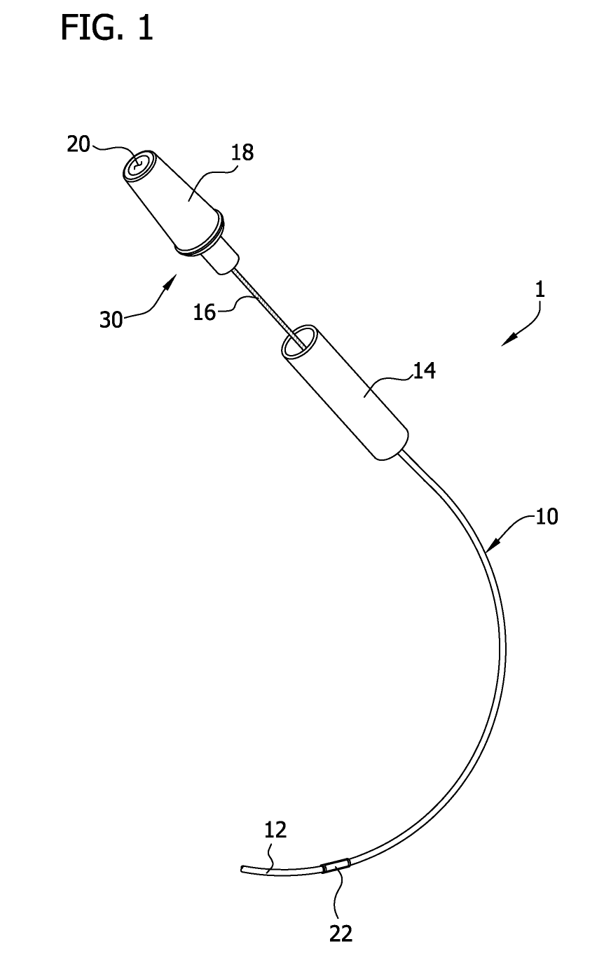

Referring now to the drawings and, in particular, FIG. 1, an intubation device is generally indicated at 1. The intubation device includes a stylet 30 and tube 10. The stylet 30 includes a wire 16 and hub 18. The stylet wire 16 is located within the lumen formed by the tube 10 after application of the device to the patient.

The tube 10 includes a weighted bolus 12 at its distal end that helps maintain intubation. The bolus may be weighted by tungsten or any other suitable material. As used herein, the “distal end” of the tube is the end of the tube that is located in the target organ of the patient after application. The “proximal end” of the tube is the end of the tube that is external to the patient after application of the tube and to which, for example, a nutrition, water or air source may be connected.

The distal end of the tube 10 includes a pair of opposed openings 22 or “feeding ports” (one shown). A connector 14 is attached to the proximal end of the tube 10 for connection to the nutrition, water or air source. The connector 14 may be integral with the tube or may be attached by adhesion or by friction-fit.

The hub 18 includes a port 20. However, as the clinician is not required to activate a lubricant before removal of the stylet wire 16, in accordance with embodiments of the present invention and, as more fully explained below, the port 20 may be eliminated from the intubation device 1 without departing from the scope of the present invention.

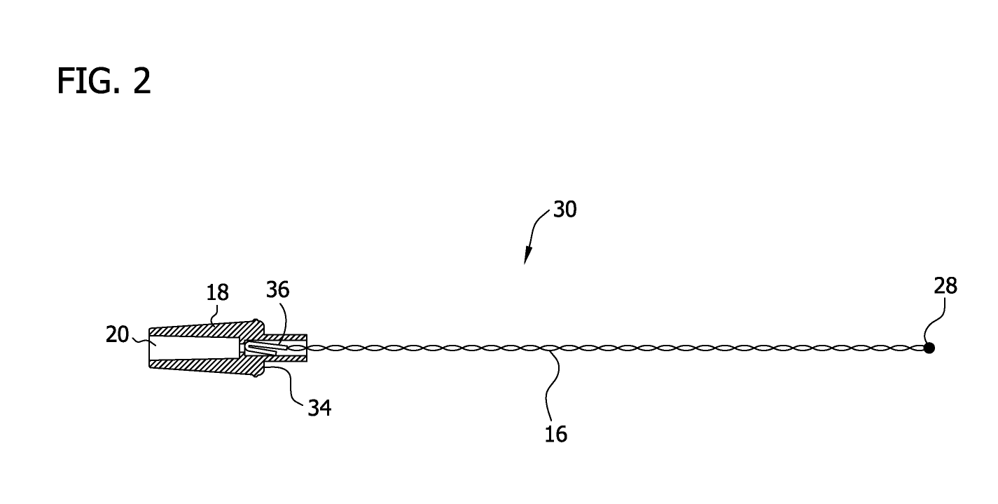

Referring now to FIG. 2, the stylet 30 includes a ball 28 at the distal end of the stylet wire 16. The ball 28 may be attached to the stylet wire by welding or other suitable means. The ball 28 is preferred over a welded spring tip used in many commercial devices as the ball causes less friction as it is removed from the tube 10, as compared to the spring tip. In addition, the ball 28 is blunt, as compared to the spring tip, and prevents the end of the wire 16 from piercing the tube 10. In the illustrated embodiment, the ball 28 is sized to be larger than the feeding ports 22 to prevent the distal end of the stylet wire from extending outside the tube 10 and possibly injuring the patient. The ball 28 may be sized smaller than the feeding ports 22 without departing from the scope of the present invention.

The stylet wire 16 is made of two individual wires that are wound as a double-helix. The two wires may be wound to about 5 twists per inch. The double-helix design reduces the surface area of the wire 16 that contacts the inside surface of the tube 10 as the wire is withdrawn from the tube. Because there is less area in contact between the two surfaces, the friction between the tube and wire is reduced and less force is required to remove the wire from the tube.

A tail 36 is attached to the proximal end of the wire 16 by suitable means such as welding. Alternatively, the tail 36 may be integral with and form part of the wire 16 itself. The tail 36 is generally U-shaped. The proximal end of the wire 16 is connected to the hub 18 in a suitable manner such as by insert-molding the tail 36 in the hub.

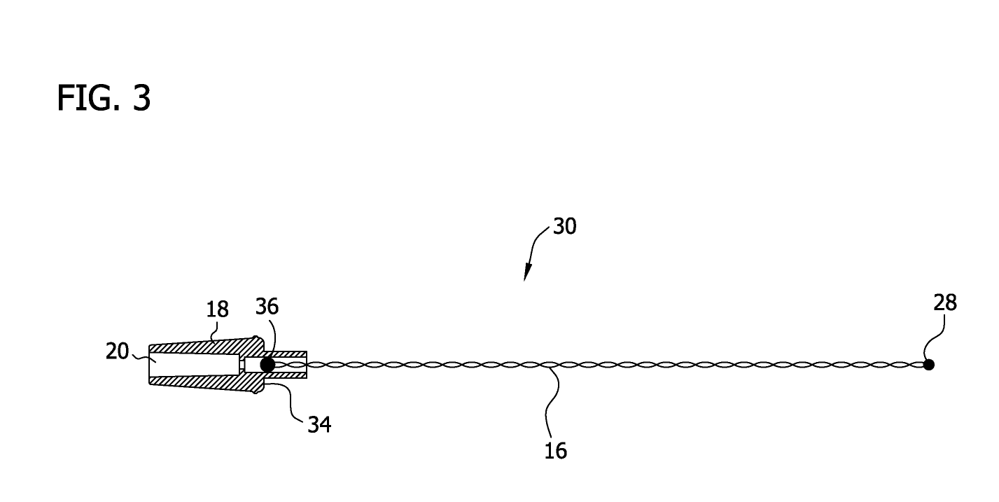

In an alternate stylet (FIG. 3), the tail is replaced by a ball 36. The ball may be insert-molded into the hub to attach the proximal end of the wire 16 to the hub.

The body of the tube 10 includes a mixture of a polymer composition and a lubricant. The lubricant used in accordance with the present invention is selected to provide inherent lubricity to the surface of the body, i.e., the lubricant does not need to be activated, for example, by contact with water or an alkaline solution as with prior intubation devices. The lubricant should be acceptable for medical use and for insertion into the human body. Suitable lubricants include amide waxes, fatty esters and mixtures thereof. The amide wax lubricant may be saturated or unsaturated.

The lubricant reduces friction between the inner surface of the tube body and the stylet wire as the stylet wire is withdrawn from the lumen formed by the tube body without requiring activation (i.e., the lubricant is inherently lubricious). The lubricant also reduces friction between the tube and body conduits of the patient (e.g., nasal cavity or esophagus) as the intubation procedure is performed.

Especially preferred amide waxes comprise two or more iminocarbonyl moieties. As used herein, an iminocarbonyl moiety is a compound of Formula I

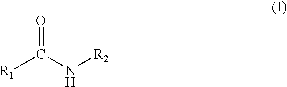

wherein R₁ and R₂ are independently hydrogen, a hydrocarbyl or substituted hydrocarbyl.

As used herein, “hydrocarbyl” describes organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 30 carbon atoms. As used herein, “substituted hydrocarbyl” moieties are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, alkynoxy, aryloxy, hydroxy, protected hydroxy, keto, acyl, acyloxy, nitro, amino, amido, nitro, cyano, thiol, ketals, acetals, esters and ethers.

Amide waxes with two or more iminocarbonyl moieties include N,N′-ethylenebisstearamide and N,N′-dioleoylethylenediamine. N,N′-dioleoylethylenediamine is an unsaturated amide wax of Formula II

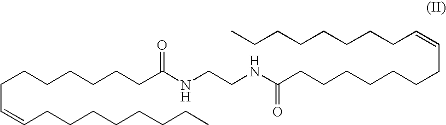

N,N′-ethylenebisstearamide is a compound similar to Formula II, but is a saturated amide wax. N,N′-ethylenebisstearamide is illustrated in Formula III

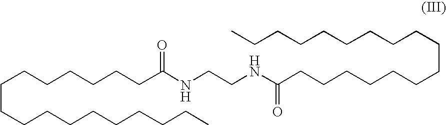

N,N′-ethylenebisstearamide may be produced by reacting two moles of stearic acid and one mole of ethylenediamine and is commercially available from Lonza Inc. (Williamsport, Pa.) under the brand name Acrawax® C. N,N′-dioleoylethylenediamine may be produced by reacting two moles of oleic acid and one mole of ethylenediamine and is commercially available from Lonza, Inc. under the brand name Glycolube® VL.

The polymer should form a homogeneous mixture with the lubricant, i.e., should form a homogeneous plasticized mixture with amide waxes and fatty esters. In addition, the polymer should not adversely react with or affect the ability of the lubricant to function as a lubricant. The polymer should be acceptable for medical use and for insertion into the human body.

The polymer composition may include a polymer resin selected from the group consisting of polyurethanes, polyvinylchlorides, polypropylenes, polyethylenes and mixtures thereof. Preferably, the polymer composition comprises a polyurethane resin. An especially well-suited polymer for use in medical tubing is a polyurethane polymer that is the reaction product of methylenediphenyl, diisocyanate, 1,4-butanediol and polytetramethylene glycol. An example of such a polymer is sold under the brand name Pellethane® by the Dow Chemical Company.

Typically, the intubation tube is manufactured by mixing the lubricant with the polymer composition, melting the mixture and molding the mixture to form the tube. It is not necessary to coat the tube with the lubricant before application as with other commercial devices. It is preferred that the tube body comprise at least about 0.1% by weight lubricant. It is more preferred that the tube body comprise from about 0.1% to about 10% by weight lubricant, from about 0.1% to about 5% by weight lubricant, from about 0.1% to about 1% by weight lubricant, or from about 0.25% to about 0.75% by weight lubricant. In one embodiment, the body comprises about 0.50% by weight lubricant.

The tube body may, optionally, include an amount of barium sulfate. Barium sulfate provides opacity to the tube body. It is preferred that the tube body comprise from about 10% to about 30% by weight barium sulfate. According to other embodiments, the tube body comprises from about 15% to about 25% by weight barium sulfate, from about 18% to about 22% by weight barium sulfate. According to another embodiment, the tube comprises about 20% by weight barium sulfate. Barium sulfate is readily incorporated into the tube body by adding it with the polymer composition and lubricant mixture during the manufacturing process.

In one embodiment, the polymer composition consists essentially of the reaction product of methylenediphenyl, diisocyanate, 1,4-butanediol and polytetramethylene glycol and the body comprises at least about 0.1% by weight N,N′-dioleoylethylenediamine.

A patient may be intubated with the intubation device according to methods commonly practiced by medical clinicians. In contrast to prior methods, the lubricant does not need to be activated by flushing water or an alkaline solution into the tube prior to withdrawal of the stylet.

Before the intubation procedure begins, the distal end of the stylet wire 16 is inserted into the proximal end of the tube 10 until the proximal end of the connector 14 contacts a stop 34 (FIG. 2) on the hub 18 of the stylet 30. The distal ends of the tube 10 and stylet wire 16 are then inserted as one piece through a body orifice (e.g., nostril or mouth), through body conduits (e.g., larynx or esophagus) and into the target body organ (e.g., stomach, intestine or trachea).

For instance, when installing a nasogastric device, the distal end of the intubation device is gently inserted into the nostril and aimed down the back toward the ear. As the bolus drops below the soft palate into the pharynx, the patient is encouraged to swallow, if possible. Giving the patient small amounts of water to sip through a straw is sometimes helpful. The practitioner then continues to gently assist the tube down the esophagus and into the stomach until the desired position is reached. In doing so, caution must be exercised not to use force. One must proceed slowly and carefully. However, if the patient coughs or cannot vocalize or shows signs of respiratory distress, this may indicate that the tube has instead entered the trachea. If this occurs, the tube must be withdrawn and inserted into the esophagus.

Before withdrawing the wire stylet, the clinician ensures that the tube has reached the desired position by one or all of the following methods: (a) auscultation, e.g. by injecting with a syringe 10-20 cc of air through tube/stylet assembly and listening for a bubbling sound in the upper left abdominal quadrant; (b) aspiration by using a syringe to withdraw a small amount of gastric contents; or, most preferably, (c) X-ray.

When the tube is properly positioned, the stylet is slowly and carefully withdrawn from the tube by applying a pulling force to the proximal end of the stylet to slide the stylet wire in a proximal direction relative to the tube. The source of nutrition or water may then be attached to the connector 14 or the connector may be capped to prevent entry of foreign objects into the tube. The tube may be taped to the patient's nose, cheek or forehead to stabilize the tube.

Tracheal intubation may be performed by direct laryngoscopy in which a laryngoscope is used to obtain a view of the glottis. A tube is then inserted through the mouth and into the trachea by direct vision. This technique can usually only be employed if the patient is under general anesthesia or has received local or topical anesthesia to the upper airway structures. Other techniques include rapid sequence induction for patients under anesthesia or use of a flexible endoscope or video laryngoscope for patients who are awake and under local anesthesia.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above methods without departing from the scope of the invention, it is intended that all matter contained in the above description and shown in the accompanying figures shall be interpreted as illustrative and not in a limiting sense.

What is claimed is:

1. An intubation device for intubation of a patient, the device comprising:
a tube comprising a body that forms a lumen, the body comprising a mixture of a polymer composition and lubricant, the lubricant providing inherent lubricity to the surface of the body; and
a stylet comprising a stylet wire adapted and sized for insertion into and removal from the lumen, the stylet wire providing rigidity to the tube upon insertion into the lumen.

2. An intubation device as set forth in claim 1 wherein the lubricant is selected from the group consisting of amide waxes, fatty esters and mixtures thereof.

3. An intubation device as set forth in claim 2 wherein the lubricant comprises an amide wax.

4. An intubation device as set forth in claim 3 wherein the lubricant consists essentially of an amide wax.

5. An intubation device as set forth in claim 3 wherein the amide wax comprises two or more iminocarbonyl moieties.

6. An intubation device as set forth in claim 5 wherein the amide wax is saturated.

7. An intubation device as set forth in claim 5 wherein the amide wax is unsaturated.

8. An intubation device as set forth in claim 3 wherein the amide wax is selected from the group consisting N,N′-ethylenebisstearamide, N,N′-dioleoylethylenediamine and mixtures thereof.

9. An intubation device as set forth in claim 3 wherein the amide wax comprises N,N′-dioleoylethylenediamine.

10. An intubation device as set forth in claim 1 wherein the polymer composition comprises a polymer resin selected from the group consisting of polyurethanes, polyvinylchlorides, polypropylenes, polyethylenes and mixtures thereof.

11. An intubation device as set forth in claim 10 wherein the polymer composition comprises a polyurethane resin.

12. An intubation device as set forth in claim 11 wherein the polyurethane is the reaction product of methylenediphenyl, diisocyanate, 1,4-butanediol and polytetramethylene glycol.

13. An intubation device as set forth in claim 1 wherein the body comprises at least about 0.1% by weight lubricant.

14. An intubation device as set forth in claim 1 wherein the body comprises from about 0.1% to about 10% by weight lubricant.

15. An intubation device as set forth in claim 1 wherein the body comprises from about 0.1% to about 5% by weight lubricant.

16. An intubation device as set forth in claim 1 wherein the body comprises from about 0.1% to about 1% by weight lubricant.

17. An intubation device as set forth in claim 1 wherein the body comprises from about 0.25% to about 0.75% by weight lubricant.

18. An intubation device as set forth in claim 1 wherein the body comprises about 0.50% by weight lubricant.

19. An intubation device as set forth in claim 1 wherein the body further comprises barium sulfate.

20. An intubation device as set forth in claim 1 wherein the polymer composition consists essentially of the reaction product of methylenediphenyl, diisocyanate, 1,4-butanediol and polytetramethylene glycol and the body comprises at least about 0.1% by weight N,N′-dioleoylethylenediamine.

21. An intubation device as set forth in claim 1 wherein the stylet wire comprises two wires wound in a double-helix.

22. An intubation device as set forth in claim 1 wherein the stylet further comprises a ball at a distal end of the stylet wire.

23. An intubation device as set forth in claim 1 wherein the stylet further comprises a hub and the stylet wire is insert-molded into the hub.

24. An intubation device for intubation of a patient, the device comprising:
a tube comprising a body that forms a lumen, the tube being sized to deliver nutrients, water or air from a source external to a patient, through a body orifice and into a body organ of the patient, the body comprising a mixture of a polymer composition and lubricant, the lubricant providing inherent lubricity to the surface of the body, wherein the polymer composition and lubricant are suitable for use in the human body.

25. An intubation device as set forth in claim 24 wherein the tube is sized to extend from the source of nutrients, water or air, through a nostril and into the stomach of the patient.

26. A method for intubating a patient, the method comprising:
inserting an intubation device through a body orifice and into a body organ of a patient, the device comprising:
a tube comprising a body that forms a lumen, the body comprising a mixture of a polymer composition and a lubricant, the lubricant providing inherent lubricity to the surface of the body; and
a stylet comprising a stylet wire received within the lumen having a proximal end and a distal end;
removing the stylet wire from the lumen by applying a pulling force to the proximal end of the stylet wire to slide the stylet wire in a proximal direction relative to the tube.

27. A method for intubating a patient as set forth in claim 26 wherein the orifice is a nasal passage and the body organ is the stomach.

28. A method for intubating a patient as set forth in claim 26 wherein the lubricant is selected from the group consisting of amide waxes, fatty esters and mixtures thereof.

29. A method for intubating a patient as set forth in claim 28 wherein the lubricant comprises an amide wax.

30. A method for intubating a patient as set forth in claim 29 wherein the lubricant consists essentially of an amide wax.

31. A method for intubating a patient as set forth in claim 29 wherein the amide wax comprises two or more iminocarbonyl moieties.

32. A method for intubating a patient as set forth in claim 31 wherein the amide wax is saturated.

33. A method for intubating a patient as set forth in claim 31 wherein the amide wax is unsaturated.

34. A method for intubating a patient as set forth in claim 29 wherein the amide wax is selected from the group consisting N,N′-ethylenebisstearamide, N,N′-dioleoylethylenediamine and mixtures thereof.

35. A method for intubating a patient as set forth in claim 29 wherein the amide wax comprises N,N′-dioleoylethylenediamine.

36. A method for intubating a patient as set forth in claim 26 wherein the polymer composition comprises a polymer resin selected from the group consisting of polyurethanes, polyvinylchlorides, polypropylenes, polyethylenes and mixtures thereof.

37. A method for intubating a patient as set forth in claim 36 wherein the polymer composition comprises a polyurethane resin.

38. A method for intubating a patient as set forth in claim 37 wherein the polyurethane is the reaction product of methylenediphenyl, diisocyanate, 1,4-butanediol and polytetramethylene glycol.

39. A method for intubating a patient as set forth in claim 26 wherein the body comprises at least about 0.1% by weight lubricant.

40. A method for intubating a patient as set forth in claim 26 wherein the body comprises from about 0.1% to about 10% by weight lubricant.

41. A method for intubating a patient as set forth in claim 26 wherein the body comprises from about 0.1% to about 5% by weight lubricant.

42. A method for intubating a patient as set forth in claim 26 wherein the body comprises from about 0.1% to about 1% by weight lubricant.

43. A method for intubating a patient as set forth in claim 26 wherein the body comprises from about 0.25% to about 0.75% by weight lubricant.

44. A method for intubating a patient as set forth in claim 26 wherein the body comprises about 0.50% by weight lubricant.

45. A method for intubating a patient as set forth in claim 26 wherein the body further comprises barium sulfate.

46. A method for intubating a patient as set forth in claim 26 wherein the polymer composition consists essentially of the reaction product of methylenediphenyl, diisocyanate, 1,4-butanediol and polytetramethylene glycol and the body comprises at least about 0.1% by weight N,N′-dioleoylethylenediamine.

47. A method for intubating a patient as set forth in claim 26 wherein the stylet wire comprises two wires wound in a double-helix.

48. A method for intubating a patient as set forth in claim 26 wherein the stylet further comprises a ball at a distal end of the stylet wire.

49. A method for intubating a patient as set forth in claim 26 wherein the stylet further comprises a hub and the stylet wire is insert molded into the hub.
